(19) United States
(12) Patent Application Publication
(10) Pub. No.: US 2006/0205057 A1
(43) Pub. Date: Sep. 14, 2006

(54) Microfluidic microarray with high surface area active regions

(76) Inventors: Danial D. M. Wayner, Ottawa (CA); Gerardo A. Diaz-Quijada, Montreal (CA)

Correspondence Address: ADE & COMPANY INC., P.O. BOX 28006 1795 HENDERSON HIGHWAY, WINNIPEG, MB, R2G1P0, CA

(21) Appl. No.: 11/357,412

(22) Filed: Feb. 21, 2006

Related U.S. Application Data
(60) Provisional application No. 60/654,517, filed on Feb. 22, 2005.

(30) Foreign Application Priority Data
Feb. 18, 2005 (CA) 2,497,577

(51) Int. Cl. C12M 1/00

(52) U.S. Cl. 435/283.1

(57) ABSTRACT

Described herein is a method of providing three dimensional structures in a fluidic channel. The assay system comprises a channel, for example, a microfluidic channel, and a plurality of posts mounted to a support and arranged for insertion into the channel. The posts may be treated such that the posts are microporous, thereby greatly increasing the surface area of the posts. The posts are then treated with an agent that either activates the surface of the posts or facilitates immobilization of biomolecules of interest on the surface of the posts.

### PRIOR APPLICATION INFORMATION

This application claims the benefit of Canadian Patent Application 2,497,577, filed Feb. 18, 2005 and of U.S. Provisional Application 60/654,517, filed Feb. 22, 2005.

### BACKGROUND OF THE INVENTION

Microarray technologies are well established for the parallel analysis of a wide range of bioactive components such as DNA, proteins and other small molecules. The main drawback of this technology is the time required for capture of often dilute probe molecules in solution. In addition, relatively large sample volumes are required (on the order of tens of microlitres). Thus, analyses typically require several hours of incubation time to ensure that all probe molecules are captured quantitatively (i.e., mass transport limited). On the other hand, microfluidic technologies, which also are well established, have been used primarily for electrophoretic or other types of separations techniques followed by in-channel detection (e.g. electrochemical or fluorescence). While the volumes sampled are quite small (on the order of tens of nanolitres) the diversity of analyses possible in a single experiment are quite limited. The problem is that at this time, there are no commercial devices that combine the best attributes of microarrays (high throughput) and microfluidics (small volumes, short analysis times) in a single device.

A number of researchers have attempted to combine microfluidics and microarrays by printing a microarray on a surface (e.g. glass) then overlaying a microfluidic channel so that the microarray is exposed to the flowing solution on one surface of the fluidic channel (FIG. 1).^{1.2}

However, these methods and devices are limited in the following ways: (1) Diffusion to the walls of the channel requires several seconds in typical fluidic devices. These devices have channels with dimensions on the order of 50-100 microns requiring about 10 seconds for diffusion of the material in solution to the nearest wall. (2) The current devices use surfaces that are dense and flat (i.e. non-porous). Thus, total amount of material that can be captured is limited to the two dimensional surface area of the active region, typically on the order of 10¹⁰ molecules/cm². As a consequence, some degree of porosity is desirable since it increases the surface area and therefore the amount of material that can be captured. However, the pore size should be sufficiently large to allow biomolecules to access the inner surface of the pore. Some devices confine the analyte close to the active surface by injecting the analyte solution and a buffer into two different microchannels that are part of a T-junction configuration. Confinement is achieved by the high degree of laminar flow which prevents mixing in microfluidic devices.³ While this can be effective, the approach drastically reduces the sample volume and requires much longer contact times and/or preconcentration of the sample.

### SUMMARY OF THE INVENTION

According to a first aspect of the invention, there is provided an assay device comprising:

a channel for flowing a liquid sample therethrough; and

a plurality of three dimensional structures in the channel.

The three dimensional structures may have a microporous surface.

The microporous surface may be activated for attachment of at least one biomolecule.

The channel may have electroosmotic properties.

According to a second aspect of the invention, there is provided a microchannel having a microporous surface for flowing a liquid sample therethrough.

### BRIEF DESCRIPTION OF THE DRAWINGS

FIG. 1. Standard approach to building microfluidic channels around pre-spotted microarrays on glass.

FIG. 2. Fabricated posts in polydimethylsiloxane (PDMS). Circular posts arranged in line with 5 micron diameter (left) and 10 micron diameter (right). The posts are 50 microns in height.

FIG. 3. Reaction of the porous oxidized PDMS with APTES.

FIG. 4. Schematic of a microfluidic device with an embedded array of posts. The coloured rectangles represent the regions containing the posts.

FIG. 5. Fluorescence signal from a plug of solution containing goat-antirabbit IgG (left) and sheep-antimouse IgG (right) labeled with Cy3 flowing over posts that have been modified with rabbit IgG. The detector is focused on a region of the device containing 5 micron posts separated by 4 microns (edge-to-edge).

FIG. 6. Fluorescence signal from a plug of solution containing goat-antirabbit IgG (left) and sheep-antimouse IgG (right) labeled with Cy3 flowing over a region of a channel with only three walls modified with rabbit IgG. For comparison purposes, the length of the channel that was modified with the antibody was the same as in the case for the device with posts.

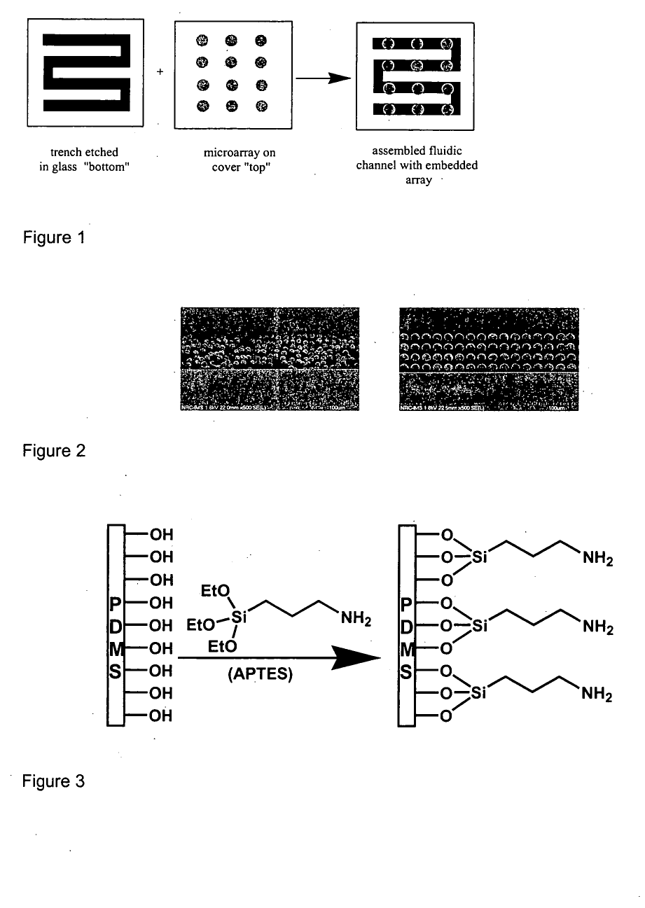

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

As will be understood by one of skill in the art, as used herein, microfluidics is the science of designing, manufacturing, and formulating devices and processes that deal with volumes of fluid on the order of nanoliters (symbolized nl and representing units of 10⁻⁹ liter) or picoliters (symbolized pl and representing units of 10⁻¹² liter). The devices themselves have dimensions ranging from millimeters (mm) down to micrometers (μm), where 1 μm=0.001 mm.

Described herein is a method of providing an improved microfluidic assay system. In some embodiments, the assay system comprises three dimensional structures in a fluidic channel. In these embodiments, the assay system comprises of a channel, for example, a microfluidic channel, and a plurality of posts mounted to a support and arranged for insertion into the channel or a plurality of posts within the channel, said posts arranged such that the posts extend outwardly from a surface of the channel. The posts and/or the channel may be treated such that the surfaces of the posts and the channel are microporous or nanoporous, thereby greatly increasing the surface area of the posts and the channel, as discussed below. Preferably, the pores are of a diameter between about 1 nm to about 200 nm. The posts are then treated with an agent that either activates the surface of the posts or facilitates immobilization of biomolecules of interest on the surface of the posts. The channel may be treated such that electroosmotic behaviour is induced, as discussed below.

In some embodiments, the posts are omitted and the biomolecules are bound to the walls of the channel. As discussed below, in these embodiments, the lack of the three dimensional structures or posts within the channel results in an increased diffusion time compared to those embodiments wherein the posts are used. As will be appreciated by one of skill in the art, this arrangement is suitable for several uses, as discussed herein.

It is of note that the three dimensional structures are referred to herein as “posts”. As will be apparent to one of skill in the art, the three dimensional structures may be of any suitable architecture or geometry for use within the invention.

Bioactive molecules or biomolecules of interest, for example, but by no means limited to DNA, peptides, carbohydrates, proteins, antibodies or any other organic, metallo-organic or inorganic molecule with biological activity or potential biological activity are then linked to the posts, either directly or via a suitable cross-linker. It is of note that in some embodiments, all of the biomolecules on a given post or a group of posts or substantially all of the posts may be identical. In other embodiments, the biomolecules on the posts may be directed to bind different regions or aspects of a specific organism or biomolecule, for example, different antibodies against different bacteria cell surface proteins or different cell surface proteins of a specific strain of bacteria. In yet other embodiments, the biomolecules may be directed against a plurality of different organisms or targets potentially found in a specific sample, for example, to bind common pathogens or compounds found in blood, urine or drinking water.

In some embodiments, the channel is treated such that the channel has a long-lived electroosmotic behaviour, as discussed below.

In the examples provided, we use posts with a density of about 10,000 posts/mm². However, the range may be from no posts (0 posts/mm²) at all (as in FIG. 6 and as discussed above) up to 400,000,000 posts/mm². Although FIGS. 5 and 6 demonstrate the advantage of reducing the diffusion time, as discussed above, the walls of the device in the active region are also microporous. If they were not, then the intensity of the signal would be so low that we would not be able to measure it over the background noise.

In use, a sample of interest is applied to a channel. It is of note that the posts, prepared as described above, may be inserted and/or fabricated into the channel prior to, during, or after sample application. As discussed below, the sample diffuses such that material present in the sample is presented to or flows past the biomolecules attached to the posts and may specifically bind thereto. As discussed below, in some embodiments, the channel may be connected to a pump. Material bound to the posts is then detected as discussed below.

Thus, the instant assay system combines the advantageous features of microarrays and microfluidics, resulting in an assay system that is capable of high throughput, has a short analysis time and requires small volumes.

In some embodiments, the three dimensional structures may be ordered, although this is not a requirement of the invention, as discussed below. The three dimensional structures increase the effective surface area and reduce the diffusion times to the nearest surface. For example, in a channel 50 microns high and 100 microns wide containing posts separated by 5 microns, the diffusion time to the nearest post is only about 30 ms compared to about 10 seconds in the absence of the posts. An example of such microfabricated arrays of posts is shown in FIG. 2.

It is of note that the channels may have dimensions in the range from a few millimetres to sub-micron for both the width and depth. For example, in some embodiments, the channel width and depth may be from about 10 microns to about 100 microns while in other embodiments, the channel height and depth may be from about 1 micron to about 1 mm.

The posts are activated for chemical modification in the following way. First, an active porous layer is grown over the posts by ozone oxidation.⁴ The porous oxidized layer is reacted with an agent that either activates the surface, for example, silanating reagents with expoxy groups, or facilitates the immobilization of biomolecules. The agent used does not need to have any specific properties other than being able to react chemically in the intended manner and suitable agents are well known by one of skill in the art. In a preferred embodiment, the agent is aminopropyl triethoxy silane (APTES) and treatment with APTES is followed by either direct reaction with the appropriate activated biomolecule or an appropriate cross-linker (e.g. gluteraldehyde or glutaric anhydride) to provide other surface functional groups suitable for direct reaction with bioactive molecules (FIG. 3). Although the present example illustrates the ability of modifying a cluster of posts with a particular biomolecule, it will be apparent to one of skill in the art that the modification of individual posts with different biomolecules can be done within this assay system, as discussed above and in the examples below. All remaining channel walls outside of the active regions have been modified using a process described below, with a reagent that induces long lived electroosmotic behaviour. It is well known that oxidation of PDMS microchannels with an oxygen plasma induces electroosmotic behaviour. However, this effect is lost within 24 hours. As will be apparent to one of skill in the art, such a short shelf-life is not suitable for a commercial device.

In preferred embodiments, it is desirable to fabricate the posts as tall as possible with the smallest diameter (i.e. aspect ratio=height/diameter) in order to increase the surface area and the rate of capture. We have been able to fabricate posts with an aspect ratio of 10. At this aspect ratio, the posts are no longer rigid and they tend to somewhat aggregate. However, it is important to note that that this is not a limitation. It should be noted that in aqueous solution, the posts have a high surface charge and will tend to repel each other and no longer aggregate. In addition, at high aspect ratios, the posts tend to be quite fragile and more difficult to fabricate or release from the silicon mould. Ideally, the posts should be fabricated with the smallest possible diameter to allow for a high density and the height of the posts would have to be decreased accordingly to maintain an aspect ratio within the desired range. For example, the posts may be between about 1 micron and about 100 microns.

As discussed above, examples of suitable biomolecules include but are by no means limited to DNA, peptides, carbohydrates, proteins, antibodies, any other organic, metallo-organic or inorganic molecule with biological or potential biological activity and the like.

As discussed above, in some embodiments, the assay system includes mechanical (or pressure) pumping as an alternative to electroosmosis. In some embodiments, pumping of fluids may be necessary to bring about molecular recognition events. As an example, consider one biomolecule is immobilized on the surface. A second biomolecule in solution is brought in contact with the first one by means of pumping. If there is molecular recognition (specific binding), then the biomolecule in solution is captured. Specific binding events are the basis for high throughput screening and diagnosis.

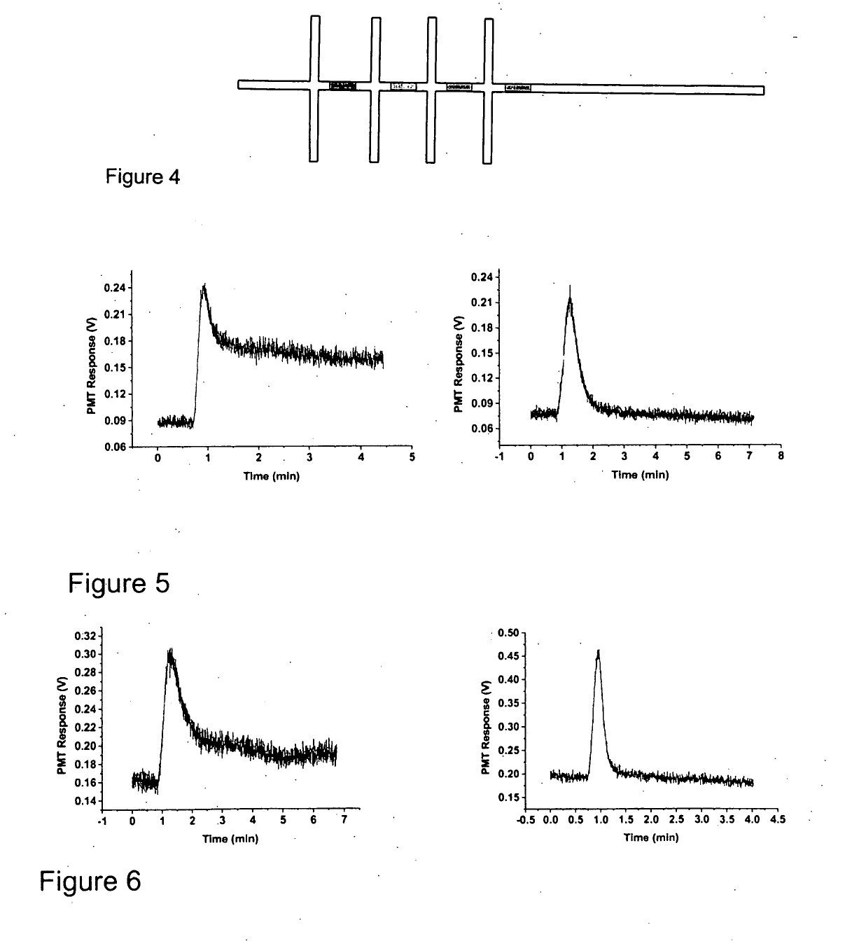

As part of a microfluidic system, this device is demonstrably superior to a device without the posts in which three of the four walls have been modified. A prototype device is shown schematically in FIG. 4. An example of the use of this device for antibody interactions is shown in FIG. 5 and FIG. 6. In this experiment, rabbit IgG was covalently immobilized on the surface and a plug of solution containing goat antirabbit IgG or sheep antimouse IgG (control experiment) was flowed over the rabbit antibody. FIG. 5 illustrates the results for the enhanced device with microposts while FIG. 6 presents the degree of capture in the absence of posts. It is clear from these figures that the capture of the antibody in the presence of microposts is much more efficient. If one considers that the device in FIG. 6 has three rather than one (typical) wall modified, the increased efficiency of the post-modified device is about a factor of 10.

As discussed above, microporousity increases surface area by adding the third dimension to the surface that captures probes, that is, the biomolecules bound to the posts for detection of material of interest within the sample. The efficiency of the binding event depends on the concentration of available surface sites and having an effective surface area that is, due to porosity, about 100 times the dense flat surface also provides an additional advantage. There also is an increase in sensitivity since the number of fluorescent sites per unit area is higher so background fluorescence interferes less.

Diffusion limitations are minimized in the presence of a high density of posts. In this situation, biomolecules in solution have shorter diffusion distances before encountering biomolecules that have been immobilized on the surface. This is well described by Fick's laws of diffusion. Since the devices operate under laminar flow conditions (i.e. no turbulent mixing, only diffusion perpendicular to the direction of flow) the diffusion time scales proportionally to the square of the distance between reactive surfaces (i.e. not linear so the enhancement is very large).

The preferred mode of transport in microfluidic devices is based on a phenomenom known as electroosmosis. This effect is induced by the presence of charges at the surface inside microchannels. Traditionally, glass has been the material of choice in the fabrication of microchannels and its silanol groups are acidic enough to provide a negative charge on the surface. Poly(dimethylsiloxane) is an inexpensive material for the fabrication of microfluidic devices. However, this polymer is inherently electrically neutral.

As discussed above, surface oxidation using known methods such as UV/ozone and oxygen plasma generators may be employed to induce the formation of a significant number of silanol groups. Unfortunately, the surface oxidation process is rather short-lived. Typically, electroosmosis is not observed after 24 oh. This is detrimental for storage and for covalent immobilization of biomolecules. The UV/ozone method requires irradiation from different directions in order to achieve a homogeneous modification of three-dimensional structures of PDMS.

However, surface oxidization with ozone followed by immediate reaction with tetraethoxysilane induces a long-lived (at least a month) electroosmotic behaviour in microchannels by generating silanol groups at the surface of the polymer. The modified surfaces may be further modified with alkoxy-silane reagents and therefore allow covalent immobilization of biomolecules at the surface. Moreover, room temperature bonding between tetraethoxysilane modified surfaces and glass may be achieved. This is very important for the fabrication of low cost poly(dimethylsiloxane)-glass hybrid microfluidic devices.

It is of note that other tetralkoxysilanes would induce the same behaviour, as would trialkoxysilanes and dialkoxysilanes albeit to a lesser extent which may be desirable or acceptable in some embodiments. Similarly, any halogenated silanating reagent, for example, silicon tetrachloride, or a tetrakis(dialkylaminosilanes) would also be suitable.

The present method for surface modification of PDMS is simple, economical and yet effective. It can easily be implemented in large commercial processes and it is suitable for homogeneous modification of three-dimensional structures such as microchannels or structures within microchannels. Not only the resultant surfaces are effective in inducing electroosmosis, but also bond to glass surfaces when brought into conformal contact. This provides a room temperature bonding method, which is essential for the fabrication of biochip microfluidic devices. A room temperature method is required in the fabrication of biochips with biomolecules such as proteins. Moreover, the resultant surfaces have a significant shelf life and they can be further modified with standard silane and/or siloxane chemistry that allows chemical immobilization of biomolecules. A shelf life greater than 24 hours is required for the chemical immobilization of biomolecules on the surface of PDMS.

As will be apparent to one of skill in the art, electroosmosis is a simple and economical method for controlling the flow of solutions within the channels. For example, valves are not required for the manipulation of the fluids. Another advantage of electroosmotic pumping is that the velocity profile across the channel is truncated compared to pressure driven pumping (which has a parabolic profile). This avoids many of the diffusional non-uniformities that are a problem in pressure driven systems.

The combination of the features described above in a single device is novel. To our knowledge such combination that increases surface area, includes a microporous region, overcomes the diffusion limitations and supports long-term electroosmotic behaviour has never been demonstrated before. In the example above, if one considers that we modified three walls rather than one (which is typical) we have achieved an increase in signal of about a factor of 10.

In another embodiment of the invention, there is provided a kit which comprises a support including one or more channels as described above. In some embodiments, the kit may include a support with three dimensional structures such as posts mounted thereon for insertion into the channel or in other embodiments, the posts may be fabricated or inserted within the channel. The kit may include the chemicals necessary for modifying the components of the kit, for example, for inducing a microporous surface on the three dimensional structures or for activating the microporous surface for attachment of the biomolecules or for inducing electroosmotic behaviour within the microchannel as discussed above.

The present device is well suited for high throughput screening in genomics and proteomics, medical diagnostics, testing of water supplies, drug discovery, and kinetic measurements of binding events. Some specific applications include:

Point of care (at home, in doctor's office) diagnostic devices evaluating the presence of acute or chronic infectious agents (including, for example, E. Coli, cholera, HIV, Hepatitis, etc.) by direct detection of an organism, its DNA, specific protein markers, antibodies or other metabolites.

Field deployable devices for first responder evaluation of chemical or biological threats in water, air or soil. In particular, a single device could be used to evaluate multiple threats as a microfluidic microarray.

High throughput screening of libraries (peptide, nucleotide, small molecule, etc.) for drug discovery.

Electronic nose applications for evaluation of unknown threats in the environment.

Flexible research tool for measuring binding kinetics of proteins in a highly parallel format.

While the preferred embodiments of the invention have been described above, it will be recognized and understood that various modifications may be made therein, and the appended claims are intended to cover all such modifications which may fall within the spirit and scope of the invention.

### REFERENCES

(1) Jia, G. M., Kuo Sheng; Kim, Jitae; Zoval, Jim V.; Madou, Marc J; Deo, Sapna K.; Daunert; Sylvia; Peytavi, Regis; Bergeron, Michel G. Proceedings of SPIE The International Society for Optical Engineering 2004, 5455, 341-352.
(2) Lee, H. J. G., T. T.; Corn, R. M. Analytical Chemistry 2001, 73, 5525-5531.
(3) Hofmann, O. V., G.; Niedermann, P.; Manz, A. Analytical Chemistry 2002, 74, 5243-5250.
(4) Diaz-Quijada, G. A.; Wayner, D. D. M. Langmuir 2004, 20, 9607-9611.

1. A microfluidic assay device comprising:
a channel for flowing a liquid sample; and
a plurality of three dimensional structures in the channel.

2. The assay device according to claim 1 wherein the three dimensional structures have a microporous surface.

3. The assay device according to claim 2 wherein the microporous surface is activated for attachment of at least one biomolecule.

4. The assay device according to claim 1 wherein the channel has electroosmotic properties.

5. A microchannel having a microporous surface for flowing a liquid sample therethrough.
